(19) United States
(12) Patent Application Publication
(10) Pub. No.: US 2026/0273133 A1
(43) Pub. Date: Sep. 17, 2026

(54) METHOD AND APPARATUS FOR PRODUCING POLYGLUTAMIC ACID FILLER

(71) Applicant: Sang Joon Yoon, Seoul (KR)

(72) Inventor: Sang Joon Yoon, Seoul (KR)

(21) Appl. No.: 19/237,501

(22) Filed: Jun. 13, 2025

(30) Foreign Application Priority Data
Mar. 17, 2025 (KR) 10-2025-0033864

(51) Int. Cl. A61L 27/26; A61L 27/56; B29C 64/106; B29K 77/00; B33Y 10/00; C08L 5/08; C08L 77/04

(52) U.S. Cl. CPC A61L 27/26; A61L 27/56; B29C 64/106; B33Y 10/00; C08L 5/08; C08L 77/04; B29K 2005/00; B29K 2077/00

(57) ABSTRACT

The present invention provides a method for manufacturing polyglutamic acid (PGA) fillers or implants comprising: preparing the main material, polyglutamic acid, and a reinforcing material, hyaluronic acid (HA); mixing said PGA and said HA together with a solvent containing water to prepare a mixed solution; feeding said mixed solution to a hot extrusion device; extruding said mixed solution through a nozzle of said hot extrusion device; and sterilizing said extruded mixed solution.

### TECHNICAL FIELD

The present invention relates to a method and apparatus for producing polyglutamic acid (PGA) fillers and implants. More specifically, the invention relates to a method and apparatus for manufacturing polyglutamic acid fillers and implants in a desired shape more quickly and efficiently using a hot extrusion device.

### BACKGROUND ART

Fillers and implants related to skin aesthetics are typical medical products used to improve or restore the skin. Each product has a specific purpose and may have different application methods and effects on the skin.

First, fillers are used to add volume, reduce wrinkles, and create smoother facial contours. For example, different types of fillers use substances such as hyaluronic acid (HA), calcium hydroxylapatite (CaHA), and poly-L-lactic acid (PLLA). Generally, fillers are injected directly into the skin using a syringe, and they offer advantages such as short procedure time, minimal side effects, and fast recovery time.

On the other hand, implants are devices or materials that are implanted into the skin in a more permanent form than fillers and can be used to add volume to a specific area or to enhance the shape. Implants are usually solid materials made of man-made materials such as silicone or Gore-Tex. Implants are usually inserted through incisions and, unlike fillers, require a surgical approach, thus requiring a recovery period after surgery. Implants can provide permanent or long-lasting results but can be replaced or removed if necessary.

Fillers and implants that are widely used today are hyaluronic acid fillers, which use chemical crosslinkers such as 1,4-Butanediol diglycidyl ether (BDDE) and divinyl sulfone (DVS) to adjust viscoelasticity, which is their core function. However, these crosslinkers can cause toxicity and adverse effects if not fully removed from the body.

Therefore, there is a need for a new filler and implant manufacturing method that enhances durability and stability while eliminating the risk of adverse effects.

### PRIOR ART DOCUMENTS

### Patent Documents

(Patent Document 0001) Korean Published Patent No. 10-2018-0057512

### SUMMARY OF INVENTION

### Technical Problem

The present invention aims to provide a method and apparatus for manufacturing fillers and implants that do not have any side effects and can provide sufficient hydration and volumizing effects.

Additionally, the present invention aims to provide a method and apparatus for manufacturing polyglutamic acid fillers and implants in the desired shapes with high homogeneity more rapidly and efficiently using a hot extrusion device.

Furthermore, the present invention aims to manufacture highly uniform polyglutamic acid fillers and implants using 3D printing technology and photo-crosslinking.

The problems solved by the present invention are not limited to those mentioned above, and other technical problems not mentioned will be apparent to those skilled in the art based on the following description.

### Solution to Problem

According to one embodiment of the present invention, a method for manufacturing polyglutamic acid (PGA) fillers or implants may provide the method of manufacturing PGA fillers or implants that comprises: preparing the main material, polyglutamic acid, and a reinforcing material, hyaluronic acid (HA); mixing said PGA and said HA together with a solvent containing water to prepare a mixed solution; feeding said mixed solution to a hot extrusion device; extruding said mixed solution through a nozzle of said hot extrusion device; and sterilizing said extruded mixed solution.

Further, said PGA may comprise alpha (a) PGA or gamma (γ) PGA.

Further, in the step of extruding said mixed solution, the diameter of said nozzle may be maintained at 0.2 mm to 2 mm.

Further, said high-temperature extruder may maintain said mixed solution at a temperature of 80° C. to 120° C. in the step of extruding said mixed solution.

Further, the step of preparing said HA may further comprise the step of reacting said HA with a methacrylate group (MA), which is a photoreactive group, to modify said HA into HA-methacrylate (HAMA).

The method may further comprise, after the step of extruding said mixed solution, the step of cooling the extruded mixed solution to a temperature below 80° C., and the step of further mixing a photoinitiator into the extruded mixed solution and curing the mixed solution by photo-crosslinking using ultraviolet or visible light.

The method may further comprise the step of layering said extruded mixed solution using a 3D model comprising the porous structure of the PGA implant to print it in the form of a 3D structure.

Further, said hot extrusion device may comprise a 3D bioprinter.

### Effects of Invention

According to the present invention, a method and apparatus for manufacturing fillers that have no side effects and can provide sufficient hydration and volume effects can be provided.

Additionally, the present invention may provide a method and apparatus for manufacturing polyglutamic acid fillers and implants in the desired shapes with high homogeneity more rapidly and efficiently using a hot extrusion device.

Furthermore, the present invention enables the manufacture of highly uniform polyglutamic acid fillers and implants using 3D printing technology and photo-crosslinking.

The effects of the present invention are not limited to those mentioned above, and other effects not mentioned will be apparent to those skilled in the art based on the following description.

### BRIEF DESCRIPTIONS OF DRAWINGS

FIG. 1 is a flowchart illustrating a method for manufacturing PGA fillers according to one embodiment of the present invention.

FIG. 2 is a flowchart illustrating a method for manufacturing PGA implants according to one embodiment of the present invention.

FIG. 3 is a flowchart illustrating a method for manufacturing fillers and implants using a mixed solution of PGA and HA according to one embodiment of the present invention.

FIG. 4 is a flowchart illustrating a method for manufacturing fillers and implants using a mixed solution of PGA and gelatin according to one embodiment of the present invention.

FIG. 5 is a block diagram illustrating the configuration of a hot extrusion device for manufacturing PGA fillers or implants according to one embodiment of the present invention.

### DESCRIPTION OF EMBODIMENTS

The invention will be described in detail hereunder with reference to the accompanying drawings to allow those skilled in the relevant technical field to implement it easily. However, the invention may be implemented in many different forms and is not limited to the embodiments described herein.

The terminology used herein is intended to describe embodiments and is not intended to limit the invention. As used herein, singular terms also include their plural forms unless otherwise specified.

As used herein, the terms “comprises” and “comprising” do not exclude the presence or addition of one or more other components, steps, operations, and/or elements beyond those that are mentioned.

Furthermore, in describing the present invention, detailed description of the related prior art is omitted if it is deemed that such detailed description would obscure the essence of the invention.

Hereinafter, embodiments according to the present invention will be described in detail with reference to the accompanying drawings. The composition of the present invention and the effects of its operation will be clearly understood from the detailed description below.

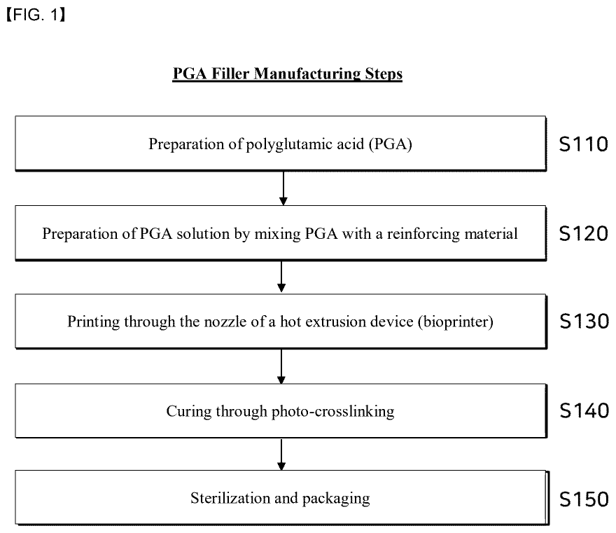

FIG. 1 is a flowchart illustrating a method for manufacturing PGA fillers according to one embodiment of the present invention.

First, the main material polyglutamic acid (PGA) is prepared. (S110)

PGA is mainly produced by microbial fermentation, in which microorganisms such as Bacillus subtilis synthesize polymers based on glutamic acid. This process is environmentally friendly and allows for mass production. For this process, microorganisms are cultured in a fermentation tank, and glutamic acid is supplied under specific conditions to generate PGA. The PGA produced during the fermentation process then undergoes a separation and purification process to remove impurities and obtain high-purity PGA.

PGA is a polymer made by polymerizing glutamic acid, an amino acid, and is widely used in the cosmetics and beauty industry for its moisturizing and anti-aging effects. Notably, it has been gaining attention in skin care products because it has superior water retention capabilities than hyaluronic acid (HA). PGA can retain about four to five times more water than HA, which is currently used as an ingredient in fillers, making it highly effective in hydrating the skin and preventing moisture loss. PGA also forms a protective layer on the surface of the skin, shielding it from external stimuli, strengthening the skin barrier function, and effectively preventing and reducing wrinkles. Additionally, with antioxidant properties, PGA helps reduce skin damage caused by free radicals and mitigate signs of aging. Because it is derived from a naturally occurring amino acid, it gets readily absorbed and broken down by the body and is very unlikely to irritate sensitive skin. The present invention aims to manufacture fillers or implants using PGA with these advantages.

Furthermore, PGA may include alpha (α) PGA or gamma (γ) polyglutamic acid. Alpha and gamma PGA (α, γ-polyglutamic acid) is a polymeric substance formed by the bonding of the amino acid glutamic acid, which is classified into alpha (α) or gamma (γ) depending on how the glutamic acid bonds. These two forms each have distinct characteristics based on their bonding structures and are utilized in various fields, including cosmetics, medicine, and agriculture. Alpha (α) PGA is formed by the bonding of the α-carboxyl group and the amino group of glutamic acid. α-PGA is mainly involved in protein synthesis in vivo and is mainly used for physiological and biological purposes. Meanwhile, gamma (γ)-PGA is formed by the bonding of the γ-carboxyl group and the amino group of glutamic acid. γ-PGA is primarily produced by microorganisms, is highly water-soluble, and is applicable in fields such as hydration, agriculture, and pharmaceuticals. γ-PGA can also attract and retain more water than HA, making it highly effective in providing hydration and preventing moisture loss in the skin, particularly for maintaining moisture in dry skin. γ-PGA also forms a protective barrier on the skin surface, helping to retain moisture and protect against external irritants.

Next, a PGA solution can be prepared by mixing reinforcing materials. (S120)

Since PGA is highly soluble in water, it can be dissolved, for example, in distilled water or phosphate buffered saline (PBS) to create a base solution, typically starting at a concentration of 5 to 20% (w/v), for example, which can be adjusted based on viscosity and printing compatibility. Additionally, the molecular weight can be adjusted according to the intended use, generally ranging from 50 to 2,000 kDa.

As a reinforcing material, at least one of gelatin, alginate and HA (HA) can be mixed with a solvent containing water to prepare a PGA mixed solution. These reinforcing materials can be used to add moisturizing and antioxidant properties, as well as safety and stabilizing components. More specifically, gelatin can be added for heat-induced reversible gelation and is effective for volume restoration and elasticity enhancement, often providing a short-lasting, natural-looking effect. The specific weight can be set at about 2 to 5% (w/v). Alginate, on the other hand, can be used to enhance strength by ionic crosslinking (calcium ions, etc.), and can effectively provide natural volume in regenerative medicine due to its fast-setting properties and cell growth-promoting properties. Meanwhile, HA, which has the effect of enhancing cell signaling and tissue compatibility, is the most effective filler material for hydration and volume restoration, and produces natural-looking results. It can also be mixed with stem cells, chondrocytes, dermal cells, or other cells depending on the intended purpose, with cell concentrations ranging from around 1 to 20×10⁶ cells/mL.

In addition, a crosslinking process is required to add chemical bonds between PGA molecules as a means to slow down the degradation rate and increase the stability and persistence of the filler. Crosslinking treatment can be chemical crosslinking treatment and photo-induced crosslinking (photo-crosslinking) treatment. For photo-crosslinking using light irradiation, a methacrylate group (MA), for example, can be added as a photo-functional group to the mixed solution in the mixed solution preparation step, and Irgacure 2959 or riboflavin can be added as a photoinitiator. Here, Irgacure 2959 is a biocompatible photoinitiator that activates at approximately 365 nm UV wavelength, and has the advantage of being compatible with PGA, a water-soluble polymer, as it dissolves in water easily. Riboflavin is an alternative biocompatible photoinitiator that is active at 365 to 450 nm and may be suitable for medical fillers.

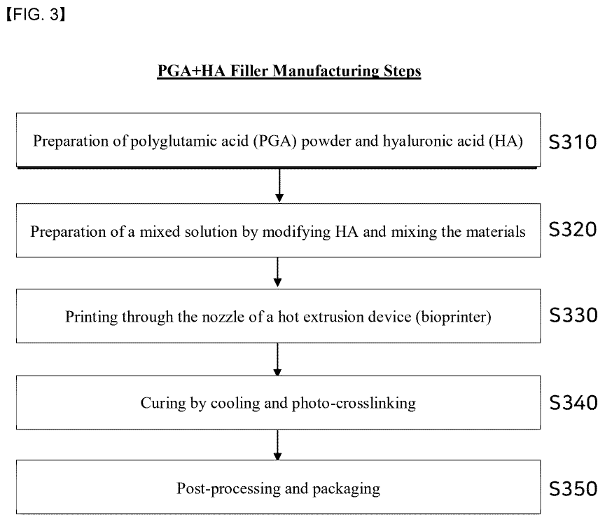

Next, the prepared mixed solution can be fed to a hot extrusion device, and the hot extrusion device can extrude the mixed solution through a nozzle. (S130) The detailed configuration of the hot extrusion device (300) will be described later with reference to FIG. 3. The hot extrusion device (300) may be configured as a 3D bioprinter, and may comprise extrusion-based, inkjet-based, or laser-based 3D printing equipment, or the like. It extrudes bioink containing the mixed solution through a fine nozzle and precisely layers the bioink based on predesigned 3D model information to print the desired shape.

In the mixed solution extrusion step, the diameter of the nozzle of the hot extrusion device (300) is maintained at 200 to 2,000 μm, ensuring the particle size is small and uniform so that it is suitable for injectable fillers and causes minimal cell damage. Furthermore, in consideration of the properties of the mixed materials, material fluidity, and cell survival among other factors, the hot extrusion device (300) can maintain the mixed solution at an appropriately high temperature, for example, 80 to 120° C., taking into account the thermal stability of glutamic acid, and maintain the pressure at 1 MPa to 5 MPa, which can be adjusted according to the diameter of said nozzle, the viscosity of the mixed solution material, and the solution concentration.

Next, the mixed solution output from the nozzle as described above can be subjected to curing by photo-crosslinking treatment. (S140) At this time, suitable light should be used according to the photoinitiator. The extruded mixed solution can be subjected to photo-curing treatment using ultraviolet light or visible light, and if the photoinitiator is Irgacure 2959, approximately 365 nm ultraviolet (UV) light can be used, whereas if the photoinitiator is riboflavin, visible light in the 365 to 450 nm range or approximately 405 nm can be used for photo-curing.

On the other hand, in the case of chemical crosslinking rather than photo-crosslinking, it is also possible to perform chemical crosslinking using 1,4-Butanediol diglycidyl ether (BDDE) or polyethylene glycol (PEG) after the mixed solution extrusion step.

The extruded and photo-cured mixed solution can then be sterilized and packaged in the desired packaging. (S150) To ensure safety, the filler undergoes a sterilization process, which involves removing pathogenic microorganisms and other contaminants. After sterilization, the filler may be packaged in an easily injectable form, such as a syringe, for example.

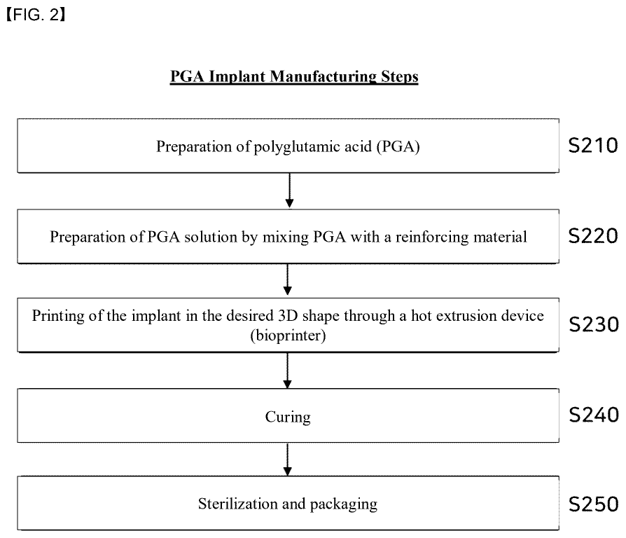

FIG. 2 is a flowchart illustrating a method for manufacturing PGA implants according to one embodiment of the present invention.

In the case of the PGA implant manufacturing method shown in FIG. 2, the configuration of steps S210, S220, and S250 is the same as the configuration of steps S110, S120, and S150 in the filler manufacturing method.

In the case of implant manufacturing, in order to print in the desired shape and form, information on the 3D model with a 3D shape is generated using CAD software or the like in advance, and the desired 3D structure can be printed using a hot extrusion device (bioprinter) using a 3D printing function. (S230) For example, a 3D model including a porous (mesh) structure for the PGA implant can be precisely printed in the form of a 3D structure by layering an extruded mixed solution using a 3D model.

The 3D printed PGA implant can then be cured. (S240) Unlike fillers, the implant does not need to be in gel form and can therefore be cured at room temperature without the need for photoinitiators or chemical crosslinkers.

FIG. 3 is a flowchart illustrating a method for manufacturing fillers and implants using a mixed solution of PGA and HA according to one embodiment of the present invention.

The method for manufacturing fillers and implants using a mixed solution of PGA and HA is generally similar to the method described with reference to FIGS. 1 and 2, but specific environmental conditions are required due to the properties of the substances.

First, PGA powder can be prepared as the main material and HA as a material to be mixed with PGA powder. (S310) Glutamic acid exists as a solid powder at room temperature, and when dissolved in water, the particle size distribution varies and precipitation may occur, making it difficult to prepare a uniform solution. HA, a biocompatible polymer with high water retention power and viscoelasticity, can be used as a matrix to disperse and encapsulate glutamic acid particles, thereby enabling the use of a hydrogel encapsulation process. Accordingly, HA can be mixed with PGA to enhance the uniformity of the particles and form a stable gel structure.

As a method of preparing a mixed solution, PGA powder is first dissolved in a small amount of water (or saline solution) to prepare a low-concentration solution, and ultrasonic treatment (sonication) can be performed at this stage to improve particle dispersion. Additionally, HA can be dissolved separately in water to prepare a viscous solution, and HA with concentrations of 1 to 5% (w/v) can generally be used. At this point, glutamic acid solution is gradually added to the HA solution to induce homogenization, during which the hydrophilic chains of HA surround the glutamic acid particles and induce initial encapsulation, and a crosslinker can be added to the mixed solution to induce bonding between HA molecules. Common chemical crosslinkers such as divinyl sulfone (DVS) or EDC/NHS may be used. Furthermore, microfluidics technology or emulsion-solvent evaporation can be used to formulate the hydrogels into micro/nano-capsules. By applying the hydrogel encapsulation process, the network structure of HA stably captures the glutamic acid particles and prevents precipitation or agglomeration, thereby improving particle uniformity, and the viscoelasticity and water retention characteristics of HA result in a soft and injectable gel-type filler formulation.

On the other hand, if a photo-crosslinking treatment is performed, HA modification is additionally required, and the glutamic acid solution and the modified HA solution are mixed together. This process will be described below.

Next, PGA solution can be prepared by modifying the HA and mixing the ingredients. (S320)

Here, HA modification is the process of making HA into a photo-crosslinkable form, such as HA-methacrylate (HAMA), by adding a photo-reactive group that can react to light, as HA is not naturally crosslinkable. For example, a methacrylate (MA) group can be selected as a photo-reactive group, and since it contains a double bond (C═C) suitable for photo-crosslinking, it can be polymerized by UV or visible light, and the methacrylate group can be photo-crosslinked by a photo-crosslinking agent, such as Irgacure 2959, by reacting with radicals generated by light to form a crosslinking network.

In this case, the process of modifying HA to a photo-crosslinkable form (e.g., HAMA) involves a chemical reaction (introduction of MA groups), which must be completed prior to hot extrusion, and a photoinitiator (e.g., Irgacure 2959), which induces crosslinking as a result of reacting to light, must be added or mixed in after hot extrusion. This is because photoinitiators such as Irgacure 2959 are sensitive to heat and can decompose or lose activity when exposed to high temperatures (>100° C.) for extended periods of time, and the stability limits of Irgacure 2959 may be exceeded at high extrusion temperatures (80 to 120° C.), resulting in reduced photo-crosslinking efficiency in case the photoinitiator is added during extrusion. Additionally, mixing the photoinitiator during extrusion requires a complex design in which light must be shone inside the extruder, which is technically difficult and costly, and if the photoinitiator is mixed in and exposed to light prior to extrusion, the crosslinking may already begin prior to extrusion, causing the mixture to undergo gelation, which is a major problem that can lead to nozzle clogging and nonuniform molding. Therefore, the photoinitiator should be added after the hot extrusion process, so that the mixture can be molded in a fluid state, and crosslinking can be induced after hot extrusion.

Next, the mixed solution can be output through a nozzle through a hot extrusion device (bioprinter). (S330)

Here, the hot extrusion device plays a role in heating and pressurizing the material in the mixed solution to mix, mold, and homogenize it, and can serve useful for mixing the glutamic acid powder and HA uniformly, molding the viscous mixture into a filler form, and manufacturing implants in the desired shape through 3D bioprinting.

At this time, the temperature condition of the hot extrusion device may be set at 80 to 120° C. in consideration of the thermal stability of glutamic acid, and the nozzle diameter may be set to be maintained in the range of 0.2 to 2 mm for injectable filler and 3D bioprinting. It is worth noting that the smaller the diameter of the nozzle, the higher the pressure required. The pressure of the hot extrusion device is maintained at 1 MPa to 5 MPa, and it can be adjusted according to the diameter size of the nozzle, viscosity, and solution concentration.

By maintaining appropriate pressure applied by a fine extrusion device under these conditions during extrusion, the mixture can be homogenized, and the extruded filler material can be output in a form suitable for photo-crosslinking. After extrusion, the mixed solution is in a viscous state that is not yet crosslinked.

Next, the extruded mixture solution may be subjected to a curing treatment through cooling and photo-crosslinking. (S340)

The extruded mixed solution is still in an uncrosslinked viscous state and can be photo-crosslinked using UV or visible light. Since photoinitiators such as Irgacure 2959 are sensitive to heat, it is first necessary to cool the mixed solution to below 80° C. after hot extrusion. For example, the extruded mixture may be cooled to room temperature (20 to 25° C.). After adding the photoinitiator solution to the cooled mixed solution and mixing it homogeneously, the photo-crosslinking treatment can be carried out, for example, by UV light (365 nm). For example, Irgacure 2959 (0.1 to 0.5% w/v) solution can be added to the cooled mixed solution and be mixed homogeneously.

In the case of PGA implant manufacturing, the extruded mixed solution can then be layered and printed into a precise 3D structure and cured at room temperature without photo-crosslinking, or the necessary curing treatment can be performed.

Next, post-processing and packaging can be performed. (S350)

After photo-crosslinking, residual photoinitiator and unreacted substances can be removed by a washing process, and sterilization can be performed before packaging the desired filler or implant according to their formulation or form.

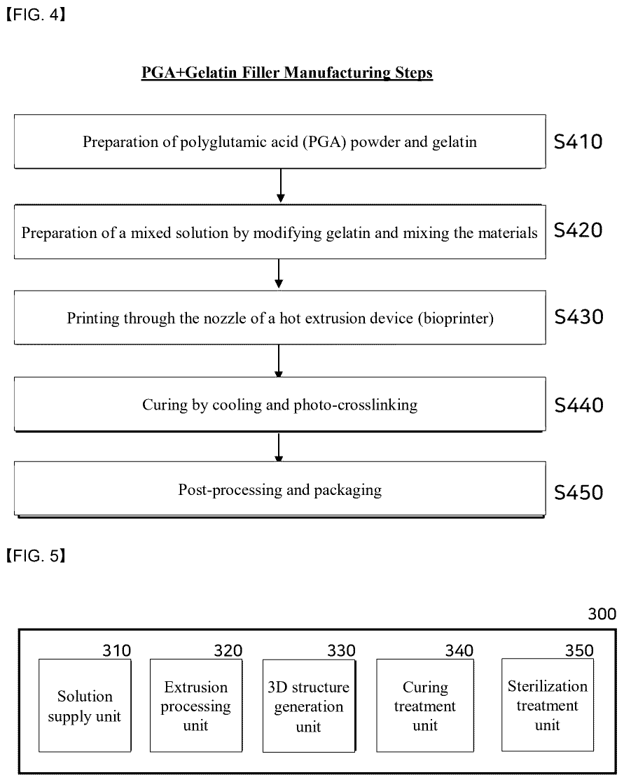

FIG. 4 is a flowchart illustrating a method for manufacturing fillers and implants using a mixed solution of PGA and gelatin according to one embodiment of the present invention.

First, PGA powder as a main material and a gelatin powder as a material to be mixed to be mixed with PGA powder may be prepared. (S410)

Gelatin is in gel form at room temperature and can be mixed with glutamic acid to improve particle uniformity and strengthen its properties as a filler. The advantages of using gelatin are that it is inexpensive, exhibits excellent biocompatibility, and makes it easy to control gel strength.

First, PGA powder is dissolved in a small amount of water (or saline solution) to prepare a low-concentration solution. The glutamic acid concentration can be started at around 1 to 5% (w/v), and the mixing ratio of glutamic acid and gelatin can be adjusted. Glutamic acid is slightly soluble in water (about 7.5 g/L at 20° C.), whereas gelatin is more soluble in hot water (40° C. to 80° C. or higher), so the temperature can be set between 4° and 80° C. for the mixing process. The glutamic acid solution is gradually added to the gelatin solution while stirring (300 to 500 rpm), so that the amino acid residues of the gelatin and the glutamic acid can be uniformly dispersed through hydrogen bonding and electrostatic interaction.

Next, a mixed solution can be prepared by mixing the photoinitiator and ingredients. (S420)

Similar to the HA modification in FIG. 3, when performing a photo-crosslinking treatment, the efficiency of the photo-crosslinking treatment can be enhanced by modifying gelatin.

Adding photoreactive groups, such as methacrylate, to gelatin creates double bonds (C═C), resulting in sites that can react with radicals generated by the photoinitiator, and as a result, methacrylate-modified gelatin (GelMA) can be produced. Photo-curing using GelMA leads to the formation of gels with high mechanical strength and durability, and photoinitiators such as Irgacure 2959 or riboflavin can be used, for example.

Accordingly, the photoinitiator (0.1 to 0.5 wt %) can be added after homogeneously mixing PGA, modified gelatin (GelMA) and solvent.

Next, the mixed solution can be output through the nozzle of a hot extrusion device (bioprinter). (S430)

At this time, the temperature of the hot extrusion device may be set to a temperature of 60 to 80° C. above the melting point of gelatin and below the decomposition temperature of glutamic acid, and the diameter of the nozzle may be set to be maintained between 0.8 and 1.2 mm or 0.2 and 2 mm, reflecting the viscosity of the gelatin mixture. It is worth noting that the smaller the diameter of the nozzle, the higher the pressure required. The pressure of the hot extrusion device is maintained at 1 MPa to 5 MPa, and it can be adjusted according to the diameter size of the nozzle, viscosity, and solution concentration.

The heat and shear force exerted by the hot extrusion device melts the gelatin and produces a molten mixture with uniformly dispersed glutamic acid particles, which can be extruded through the nozzle into a semi-solid state and be molded into the desired shape.

Next, the extruded mixture solution may be subjected to a curing treatment through cooling and photo-crosslinking. (S440)

The extruded mixed solution is cooled in a cooling chamber having a temperature of 20 to 25° C. (room temperature) or 4° C. to induce gelation of the gelatin, and then photo-crosslinking treatment may be performed using UV light or visible light (e.g., a UV lamp, wavelength: 365 nm, output: 5 to 10 mW/cm²) for about 1 to 5 minutes, during which a photoinitiator (e.g., Irgacure 2959) generates radicals, which react with the gelatin chains or with an additional crosslinking agent (GelMA) to form a crosslinking network, thereby producing a gel-like cosmetic filler with enhanced mechanical strength and durability.

In the case of PGA implant manufacturing, the extruded mixed solution can be precisely printed in the form of a 3D structure by layering an extruded mixed solution, followed by curing at room temperature without photo-crosslinking or the necessary curing treatment.

Next, post-processing and packaging can be performed. (S450)

After photo-crosslinking, residual photoinitiator and unreacted substances can be removed by a washing process, and sterilization can be performed before packaging the desired filler or implant according to their formulation or form.

FIG. 5 is a block diagram illustrating the configuration of a hot extrusion device for manufacturing PGA fillers or implants according to one embodiment of the present invention.

The hot extrusion device (300) used in the present invention may be configured to include the functions of a 3D bioprinter, and may comprise a solution supply unit (310), an extrusion processing unit (320), a 3D structure generation unit (330), a curing treatment unit (340), and a sterilization treatment unit (350).

The solution supply unit (310) is a component for supplying the prepared mixed solution described above, and it can store the mixed solution as bioink and supply it to the extrusion processing unit (320), which includes the printhead. The solution supply unit (310) may be configured to ensure continuous supply to the extrusion processing unit (320) by enabling temperature and humidity adjustments to prevent the degradation of the material during extrusion and printing.

The extrusion processing unit (320) may be configured to extrude a bioink comprising a mixed solution, cells, or the like using a nozzle at a desired temperature and pressure, and print the extruded solution by spraying or precisely stacking it through the 3D structure generation unit (330). For example, for extrusion-based printing, a screw or piston system may be used to push out the viscous bioink; for inkjet-based printing, small droplets may be sprayed using thermal or piezoelectric technology; and for laser-assisted printing, a laser may be used to transfer the bioink to the substrate. The nozzle of the extrusion processing unit (320) has micron-level precision, and in the present invention, the diameter of the nozzle can be maintained within the range of 200 to 500 μm, the temperature control device can maintain the temperature at an appropriate level in consideration of material viscosity and stability, and the pressure can be maintained between 1 and 5 MPa and be adjusted according to the diameter of the nozzle, viscosity, and solution concentration.

The 3D structure generation unit (330) can be used to print a desired shape and form in the case of manufacturing a PGA implant, and may be configured to receive information on the 3D model having a 3D shape using CAD software or the like in advance and to print the desired 3D structure shape by stacking the material using a 3D printing function. The 3D structure generation unit (330) can control the movement of the printhead and the build platform to stack the material into a 3D structure, and the movement speed and path can be programmed by software to realize the 3D shape of the implant based on the 3D model information, so that a PGA implant with a porous structure can be manufactured.

The curing treatment unit (340) can cure the extruded mixed solution through chemical crosslinking treatment or photo-crosslinking treatment. In the case of photo-crosslinking treatment, suitable light should be irradiated according to the photoinitiator used in the mixed solution, and the extruded mixed solution can be photo-cured using either UV or visible light. The photo-curing treatment can be performed by irradiating UV light in the band of about 365 nm when the photoinitiator is Irgacure 2959 and by irradiating visible light in the band of 365 to 450 nm or about 405 nm when the photo-initiator is Riboflavin. Additionally, in the case of chemical cross-linking treatment, the sterilization treatment unit (350) may be configured to perform chemical cross-linking treatment using 1,4-Butanediol diglycidyl ether (BDDE) or polyethylene glycol (PEG) after the mixed solution extrusion step.

The sterilization treatment unit (350) may be configured to sterilize the extruded PGA filler or 3D printed PGA implant. Sterilization may be performed via steam sterilization, autoclaving, or sterile filtration to remove pathogenic microorganisms or other contaminants to ensure safety. Further, after sterilization, the filler may be packaged in an easily injectable form, such as a syringe, for example.

The embodiments disclosed in the specification of the present investigation are for illustrative purposes only, and the invention is not limited these examples. The scope of the invention is to be construed in light of the following claims, and all technologies falling within the scope thereof is to be construed to be included in the scope of the invention.

### EXPLANATION OF SYMBOLS

310: Solution supply unit
320: Extrusion processing unit
330: 3D structure generation unit
340: Curing treatment unit
350: Sterilization treatment unit

1. A method for manufacturing polyglutamic acid (PGA) fillers or implants, comprising:
preparing the main material, PGA, and a reinforcing material, hyaluronic acid (HA);
mixing said PGA and said HA together with a solvent containing water to prepare a mixed solution;
feeding said mixed solution to a hot extrusion device;
extruding said mixed solution through a nozzle of said hot extrusion device;
and sterilizing said extruded mixed solution.

2. The method for manufacturing PGA filler or implants according to claim 1, wherein said PGA comprises alpha (a) PGA or gamma (γ) PGA.

3. The method for manufacturing PGA fillers or implants according to claim 1, wherein in the step of extruding said mixed solution, the diameter of said nozzle is maintained at 0.2 mm to 2 mm.

4. The method for manufacturing PGA fillers or implants according to claim 3, wherein said hot extruder maintains the mixed solution at a temperature between 80° C. and 120° C. in the step of extruding said mixed solution.

5. The method for manufacturing PGA fillers or implants according to claim 1, wherein said step of preparing HA further comprises the step of reacting said HA with a methacrylate (MA) group, which is a photoreactive group, to modify said HA into HA-methacrylate (HAMA).

6. The method for manufacturing PGA fillers or implants according to claim 5, further comprising:
cooling the temperature of the extruded mixed solution to below 80° C. after the step of extruding said mixed solution; and
further mixing a photoinitiator into the extruded mixed solution and curing the mixed solution by photo-crosslinking using ultraviolet or visible light.

7. The method for manufacturing PGA fillers or implants according to claim 1, further comprising the step of layering said extruded mixed solution using a 3D model comprising a porous structure of the PGA implant to print it in the form of a 3D structure.

8. The method for manufacturing PGA fillers or implants according to claim 1, wherein said hot extrusion device comprises a 3D bioprinter.
